(19) United States
(12) Patent Application Publication
(10) Pub. No.: US 2006/0246530 A1
(43) Pub. Date: Nov. 2, 2006

(54) Method and reagent for the specifically identifying and quantifying one or more proteins in a sample

(76) Inventors: Martin Krause, Berlin (DE); Christian Scheler, Berlin (DE); Ulrike Bottger, Berlin (DE); Hardy Weisshoff, Berlin (DE); Michael Linscheid, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/518,727

(22) PCT Filed: Jun. 13, 2003
(86) PCT No.: PCT/EP03/06391

§ 371 Date: Sep. 14, 2005

(30) Foreign Application Priority Data
Jun. 20, 2002 (DE) 102 27 599.8

(51) Int. Cl. C12Q 1/37; G01N 33/00; C07K 14/47

(52) U.S. Cl. 435/23; 436/86; 530/402

(57) ABSTRACT

The present invention relates to the MeCAT (Metal-chelate-complex-coded-affinity-tag)-method and to a reagent suitable for performing said method, which method includes a reproducible, systematic, qualitative and quantitative proteome characterization by means of non-isotope metal coded markers and—among other items—the most modem tandem methods of mass spectrometry.

The present invention relates to a method and a reagent suitable for performing said method, which method includes a reproducible, systematic, qualitative and quantitative proteome characterization by means of non-isotope metal coded markers and—among other items—the most modem tandem methods of mass spectrometry.

### BACKGROUND OF THE INVENTION

One of the most important findings of the 20^{th} century was the discovery of the DNA as the medium of all hereditary information and the elucidation of its characteristics and three-dimensional structure. The first complete DNA-sequence of an organism was published in the year 1977 by Fred Sanger. Since then, genome research has experienced extreme advance by the development of novel techniques and automated high-throughput methods, which nowadays make the sequencing of a microorganism's complete genome a routine work.

At present, biochemistry faces a novel, much greater challenge: the enormous flood of genome data being stored in gigantic electronic libraries has to be put in a functional context, thereby allowing to translate the genetic code into useful information. The realization that it is impossible to understand the complexity of biological processes only by the aid of genome analysis has introduced a further scientific branch of modem molecular cell biology, the proteome research. This is because the gene products. i.e. the proteins encoded by the genes are the actual biological effector molecules, which interfere with biological activities, control dynamic processes and perform multifaceted functions. Only they open up the opportunity to understand how the human genome and cellular processes function and how diseases arise.

As a scientific branch, proteome research (“Proteomics”) deals with the systematic identification of all proteins being expressed within a cell or tissue, and with the characterization of their essential features like e.g. amount, degree of modification, integration into multi-protein complexes, etc. Protein databases or cell maps are created, which serve the archiving of the protein sequences. Currently, many thousands of sequences and often also their investigated functions are available.

At present however, none of the applied analytical protein technologies reaches the high throughput and the level of automation of genetic engineering. Moreover, it is improbable, that a protein amplification technique analogous e.g. to PCR will be ever realizable in proteome research. What seems to be more appropriate rather is the possibility of protein enrichment, wherein the proteins of interest are extracted or enriched according to specific characteristics. It is e.g. possible to make use of physical characteristics like solubility or the capability to bind to specific ligands.

The use of the proteome analysis as a rapid and parallel method in comparison to classical protein chemistry becomes more and more common in biological fundamental research, biotechnology and medical research. It has to be expected, that this kind of analytics will be regularly established in a few years. The actual efficiency of a proteome analysis is largely dependent on the analytical methods' potential to detect and quantify the so-called “low copy” proteins, since especially they often play a crucial role in the cellular processes.

The method of proteome analysis, which is still employed most and is most reliable, is the two-dimensional gel electrophoresis (2DGE), which is subsequently followed by the sequence identification of the separated protein species. This approach reached its scientific importance for reason of the enormous progress in mass spectrometry and bioinformatics. This MS-technique, which has just recently become available and is highly sensitive, has made it possible to detect even minimal amounts of proteins and peptides—which can be made visible by conventional staining techniques—in the range of femtomoles and moreover to identify them by tandem techniques. These techniques in particular are the matrix assisted laser desorption/ionisation (MALDI) time-of-flight (TOF)-MS and the electrospray ionisation (ESI)-MS. Tandem-MS instruments like the triple-quadrupole device, ion trap, and the hybrid quadrupole time-of-flight (Q-TOF) device are routinely employed in LC-MS/MS- or nanospray experiments with electrospray ionisation (ESI), in order to generate peptide fragment ion spectra, which are suitable for the protein identification via a database sequence search.

The protein or genome data base search constitutes a tool of equal importance, which has largely contributed to the progress of proteome research. The computer search algorithms, which have been developed, are very sophisticated nowadays. Goodlett et al. were able to show that the exact mass of a peptide, in combination with limiting search criteria like the molecular weight of the protein the peptide is derived from, and the indication of the specific protease for cleaving the protein, can be sufficient for an unambiguous identification of a protein in a data base search. The high expenditure of work and the often observed non-reproducibility of the 2DGE-technique between different laboratories however make it nearly impossible to automate this method. Nowadays, there exists no analytical technique in the field of proteome research, which reaches a level comparable to genome technology. Although one is able to analyze the components of a protein mixture by means of these methods, they are neither suitable to determine the exact quantity nor the state of activity of the proteins in the mixture. Without a previous enrichment step it is virtually impossible to detect proteins being present only in very low quantities, like e.g. regulator proteins. For this reason and because of further known drawbacks of the 2DGE, research is increasingly focused on alternative methods in order to become largely independent from the 2DGE as a separation method.

Gel-free systems find the increasing interest of the proteome researchers. One can image various different gel-free systems, which are all based on the combination of two or more different chromatographic separation methods. The chromatographic separation of proteins is an essential element in any protein research and thus also constitutes an obvious method in proteome research. Due to long-standing development and optimization, chromatographic separation allows for a high reproducibility. However, even the combination of two different chromatographic methods will not allow for the resolution required in proteome research, since complex protein mixtures for reason of their characteristics are hardly to be separated into their individual, purified protein fractions. A combination between chromatography and mass spectrometry offers a further dimension of separation, the mass spectrometry, but will—when being applied to proteins—only have a very limited use advantage. As it is described in the following, this approach however will not promise success before peptides can be analyzed.

WO 00/11208 discloses an interesting alternative method for proteome analysis, which is particularly suitable for the quantitative analysis of protein expression in complex biological samples like cells and tissues, for the detection and quantification of specific proteins in complex protein mixtures and also for the quantitative determination of specific enzyme activity. It makes use of a novel class of chemical reagents, the coded affinity tags (CATs), in this case the so-called isotope-coded affinity tags (ICATs) and methods of mass spectrometry.

The ICAT-reagent consists of the affinity tag, which selectively binds to a corresponding counter-reagent in a non-covalent manner and thus allows for the separation of the affinity tag-labeled peptides or substrates from the remaining mixture by means of column chromatography. The affinity tag is coupled to a reactive group via a linker, which may optionally carry an isotope label, wherein the reactive group selectively reacts with a specific protein function.

In this way, the proteins, after being isolated from the cells, are labeled by the ICAT-reagent at specific binding sites. Here, one may e.g. have a functional group showing a specific reactivity for sulfhydryl groups, thus exclusively binding to proteins containing cysteine. From the peptide mixture obtained after enzymatic hydrolysis, one consequently isolates only the cysteine-containing peptides in a selective manner. This allows for a significant reduction of complexity of the obtained peptide mixture, since less than a tenth of all peptides, which are e.g. released from the entire yeast proteome by tryptic cleavage contain a cysteine-containing residue. A further significant advantage is that one by this approach can enrich proteins only being present in minor amounts. Despite the significant reduction of the system's complexity however, the quantification and identification of the proteins is ensured.

In order to quantitatively detect the relative amount of proteins in one or more protein samples, one now uses ICAT-reagents being isotope-coded in a different manner. Each sample is treated with an ICAT-reagent, which carries different isotope labeling, but is otherwise chemically identical. The samples, which may e.g. be derived from cell cultures of a species in different developmental phases, are unified in the following and enzymatically hydrolyzed. The labeled peptides are separated from the mixture by affinity chromatography and then fractionated by means of HPLC. A pair of peptides being identical, but being derived from different samples, is simultaneously eluted from the HPLC-column. In the mass spectrum, these peptides however do not display the same mass/charge-ratio, but differ by the characteristic mass difference of the differently isotope-labeled tags. The ratio of the relative ionic intensities of such a (CAT-labeled) peptide pair in the mass spectrum quantitatively mirrors the relative quantitative proportion of the basal proteins in the cells or tissue of origin. The peptide sequence of the ICAT-labeled peptide is then determined by fragmentation using a tandem mass spectrometer (MS/MS). The protein identification is then accomplished by a computer-aided genome or protein data base search on the basis of the recorded sequence information.

Despite all major advantages of the ICAT-method, there are still several disadvantages in its performance, which impede and complicate its use in the field of high throughput analysis. One has to employ isotopes, which significantly raise the synthesis costs of the compounds and only are of limited availability at affordable prices, thereby further restricting the method's flexibility.

It is thus the object of the present invention to provide an improved CAT-based method allowing for the employment of CAT under high throughput conditions. It is a further object to provide a CAT-reagent being specifically suitable for this method.

According to a first aspect of the present invention, this object is achieved by a method for the identification and quantification of one or more proteins in a sample containing a protein mixture. The method according to the invention comprises the steps of: a) providing a sample, which contains a mixture of proteins; b) providing a reagent for the analysis of peptides, which has the general formula
A-Y—PRG
in which A constitutes at least one functional group for the reversible, covalent or non-covalent binding to a support material, wherein said functional group comprises at least one affinity function for the selective binding to said support material, in which Y is a group comprising at least one chelate function for non-isotope metals, and in which PRG is a reactive group for the selective binding to peptides or other biomolecules to be analyzed, c) cleaving the proteins in the sample in order to produce peptides; d) coupling the peptides to the reagent of step b); e) selecting the peptides labeled in step d) under the employment of reversible binding to a support material or of affinity labeling by the binding to a support material and the removal of unbound peptides; f) releasing the bound peptides from the support material and elution from the matrix; and g) detecting and identifying the labelled peptides by means of mass spectrometry.

The method according to the present invention serves the differential investigation of the proteome of one, two or more cell, tissue or bodily fluid samples during an analysis. Moreover one may also analyze other samples containing proteins, like e.g. protein fractions of organelles or other cellular compartments. The method is a novel alternative to the ICAT-method (Isotope-coded affinity tags) being described above, and avoids some of the drawbacks of ICAT. It constitutes a novel, alternative and complementary technology for proteome research. The method of the present invention is designated “MeCAT” (Metal-chelate-complex-coded affinity-tag) in the following.

In the MeCAT method, peptide/protein samples are reacted with a MeCAT reagent, which has the following essential features:
reactive groups for the coupling to proteins/peptides or other biomolecules, in the following also designated “PRG”
at least one chelate forming complex for the (most stable possible) chelating of metals, in particular of metals being low in isotopes, in the following also designated “Y”; and
at least one affinity function (e.g. biotin) or further reactive group(s) for the coupling to support materials, solid phases or other compounds (e.g. SH-groups), in the following also designated “A”.

Instead of a labeling with different isotopes, the samples to be compared are reacted with MeCAT reagents, which differ in the chelate-bound metal ions. Subsequently, it follows e.g. an affinity purification of the labeled biomolecules, e.g. via biotin-streptavidin, or a “fishing” by means of a specific chemical reaction with a support material and subsequent release.

In the next step, the labeled biomolecules are separated by multidimensional chromatography and in the following are subjected to an on-line or off-line differential analysis with a relative quantification of the protein/peptide amount by means of mass spectrometry. The corresponding peptides of the individual samples carry—depending on the metal employed—labels of different weight and thus allow to be quantitatively assigned to individual samples, this assignment being combined with a sequence analysis (identification) of the biomolecules (peptides) by MS.

Preferred is a method according to the invention, in which the cleavage is accomplished in an enzymatic or chemical way. The cleavage can be appropriately performed by a hydrolysis under the employment of known proteases like e.g. trypsin, ASP-N-protease, pepsin, Lys-C, Glu-C, Arg-C proteinase, Asp-N endopeptidase, BNPS-scatoles, caspases, chymotrypsin, clostripain, factor Xa, glutamyl-endopeptidase, granzyme B, proline endopeptidase, proteinase K, staphylococcus peptidase A, thermolysin, thrombin, carboxypeptidases and combinations thereof. The chemical cleavage can be performed by means of partial acid hydrolysis, CNBr, formic acid, iodosobenzoic acid, NTCB (2-nitro-5-thiocyano benzoic acid), hydroxylamine, and combinations thereof.

Moreover preferred is a method according to the invention, in which the labeled peptides after their release from the support material and before their mass spectrometry analysis are chromatographically separated from each other, in particular by means of HPLC. The chromatographic technique in each case is selected according to the desired resolution.

Particularly preferred is a method according to the invention, which is characterized in that several protein and/or peptide containing samples are analyzed together. This can e.g. be achieved by the different labeling of different samples from different cell materials.

It is particularly preferred in the methods according to the invention to subject the labeled peptides to a subsequent sequencing. Using the sequence information of the labeled peptides one can perform data base searches in order to obtain hints about the basal protein.

In a further aspect, the present invention provides a method for the detection of the relative expression of proteins in a protein-containing sample, wherein said method comprises the steps of: a) providing a biological sample, which contains proteins; b) providing a reagent for the analysis of peptides, which has the general formula
A-Y—PRG
in which A constitutes at least one functional group for the reversible, covalent or non-covalent binding to a support material, in which Y is a group comprising at least one chelate function for metals, and in which PRG is a reactive group for the selective binding to peptides or other biomolecules to be analyzed, c) cleaving the proteins in the sample in order to produce peptides; d) coupling the peptides to the reagent of step b); e) selecting the peptides labeled in step d) under the employment of at least one functional group for the reversible, covalent or non-covalent binding to a support material and removal of unbound peptides; f) releasing the affinity-bound peptides from the support material and elution from the matrix; and g) detecting and identifying the labeled peptides by means of mass spectrometry; and h) measuring the relative occurrence of the differently labeled peptides as distinct peaks of ions in order to determine the relative expression of the protein, from which the affinity-labeled peptides are derived. On the basis of the analyzed expression pattern, one can draw conclusions about the different cellular states or can obtain diagnostic parameters being deduced from protein-containing samples, these results offering so far unachieved resolution.

In a further method according to the present invention, the arrangement of the groups A, X and PRG may be interchanged. Indeed, the reagent according to the invention may be present with its components being arranged in different ways, so far as all functional requirements for the performance of MeCAT are still met.

Preferably, the labeled peptides in the method according to the invention are detected by means of tandem techniques like e.g. matrix-assisted laser desorption/ionization (MALDI) time-of-flight (TOF)-TOF-MS and the electrospray ionization (ESI)-MS. For this aim, one may use an internal standard in the analysis, which can be added to the sample.

The invention comprises the MeCAT-method as well as the synthesis of the novel MeCAT-compounds. What is thus provided according to a further aspect of the present invention is a reagent for the mass spectroscopy analysis of peptides, which has the general formula:
A-Y—PRG
in which A constitutes at least one functional group for the reversible, covalent or non-covalent binding to a support material, in which Y is a group comprising at least one chelate function for metals being low in isotopes, and in which PRG is a reactive group for the selective binding of peptides or other biomolecules to be analyzed.

In an alternative reagent according to the present invention, the arrangement of the groups A, X and PRG may be interchanged. Indeed, the reagent according to the invention may be present with its components being arranged in different ways, so far as all functional requirements for the performance of MeCAT are still met.

Preferably, the function PRG is selected from a sulfhydryl-reactive group, an amine-reactive group and an enzyme substrate. It is moreover preferred, that PRG is selected from the group of an amine-reactive pentafluorophenyl ester group, an amine-reactive N-hydroxysuccinimide ester group, sulfonylhalide, isocyanate, isothiocyanate, active ester, tetrafluorophenyl ester, an acid halide and an acid anhydride, a homoserine lactone-reactive primary amine group and a carboxylic acid-reactive amine, alcohol or 2,3,5,6-tetrafluorophenyltrifluoro-acetate, a iodine acetylamide group, an epoxide, an α-haloacyl group, a nitrile, a sulfonated alkyl, an arylthiol and a maleimide.

Particularly preferred is a reagent according to the invention, in which A is selected from biotin or modified biotin, a 1,2-diol, glutathione, maltose, a nitrilotriacetic acid group, an oligohistidine or a hapten. In case of biotin, the reagent can e.g. be coupled to a streptavidin group in order to allow its convenient isolation. Particularly preferred in this context is the employment of a streptavidin-labeled column matrix or of coated beads.

In a further embodiment, A is a reactive group coupled to the support material, which reactive group can again be cleaved off the support material. Possible options for this are—among other things—disulfide bonds (S—S), which can be reduced again, thus leading to cleavage, or photosensitive bonds, which can be cleaved by exposure to light.

In a further embodiment of an inventive reagent according to the present invention, the reagent includes a chemically and/or enzymatically cleavable linker between the groups A, X and/or PRG. In general, this linker can be made up of CH₂-groups, which are located between the groups A, X and/or PRG, thereby joining these groups. One or more of the CH₂-groups can be substituted, wherein the character of the substitutions is not relevant, so far as the functions of the groups A, Y and PRG are not affected. Advantageously however, one can introduce further functions via the linkers, like e.g. the chemical and/or enzymatic cleavability mentioned above. Possible substitutions are alkyl, alkenyl and alkoxy groups, aryl groups, which may be substituted with one or more alkyl, alkenyl, alkoxy and aryl groups, acidic groups and basic groups. Moreover, double and triple bonds may be present within the linker, and heteroatoms like e.g. O, S and N may be inserted, e.g. in the form of a linker containing a disulfide group.

An essential function of the reagent according to the present invention is its chelate forming function. In preferred reagents according to the present invention, Y is selected from a macrocyclic lanthanoid chelate complex, a functionalized tetraaza-macrocycle, a polyaza-polyacetic acid, DOTA, a DOTA-derivative, NOTA, a NOTA-derivative, EDTA, DTPA-BP, DTPA, DO3A, HP-DO3A and DTPA-BMA. Particularly preferred compounds are 1,4,7,10,13,16,19,22-octaazacyclotetracosane-1,4,7,10,13,16,19,22-octaacetic acid (OTEC), and 1,4,7,10,14-17,20,23-octaazacyclohexacosane-1,4,7,10,14,17,20,23-octaacetic acid (OHEC).

The metals, which can be bound by the chelate-forming function of the reagent according to the present invention, can be selected from a large variety of metals, thereby significantly improving the flexibility when using the reagent according to the present invention. Thus, the metal bound by the chelate complex can be selected from Ag, Al, As, Au, Be, Cd, Ce, Co, Cr, Cu, Dy, Er, Eu, Fe, Gd, Hg, Ho, In, La, Li, Lu, Mn, Na, Nd, Ni, Pb, Pr, Rb, Rd, Sb, Sm, Sn, Tb, Tl, Tm, V, W, Y, Yb and Zn. According to the invention, the chelate-forming group can be labeled with several different metals.

A further aspect of the present invention relates to the use of the reagent according to the invention for the detection of peptides in a biological sample and/or the determination of the relative expression of proteins in a protein-containing sample. In this context, the biological sample can be a sample taken directly or a pre-fractionated sample for the differential investigation of the proteome of one, two or more cell, tissue or bodily fluid samples. Also investigated however can be other protein-containing samples, like e.g. protein fractions from organelles or other cellular compartments. The method is preferably applied in the course of diagnosing or monitoring the disease of an animal, in particular the human, by detecting the relative expression of proteins in a protein-containing sample taken from the animal. By the analysis and elucidation of differentially expressed proteins, one can draw conclusions about proteins being involved in diseases on a cellular level, which proteins may serve as targets for therapeutic substances or may be useful for the diagnosis and monitoring of a therapy.

A further aspect of the present invention relates to an analysis set (kit) for diagnosis, containing at least one reagent according to the present invention together with further substances and/or enzymes suitable for the detection of peptides in a biological sample and/or the determination of the relative expression of proteins in a protein-containing sample; in particular containing an internal standard. By means of this kit, one can e.g. perform a proteome labeling, the products of which can then be sent to a central analytical laboratory for analysis by mass spectrometry.

In a further variant of the method according to the invention, one may consider the employment of radioactive metal ions, which allows for a particularly sensitive detection, e.g. by scintillation counting. The respective ions are very familiar to the expert in the field of radiochemistry and may be gathered from any common chemistry textbook, such as for example the Rompp-Lexikon Chemie, 10^{th} edition, Thieme Verlag, Stuttgart.

From the view of a chemist, the entirety of possibilities for a rapid quantitative protein analysis or analysis of protein functions is by far not exhausted by the ICAT-method. The basic idea of the class of reagents presented herein is the clever combination of three different functions in one molecule; i) the possibility to specifically bind to a protein, ii) the possibility to easily separate labeled peptides from unlabelled peptides after enzymatic or chemical hydrolysis, and iii) the possibility to relatively quantify peptide pairs derived from different samples (e.g. from cells of a species in different developmental phases) via the mass difference of corresponding peptides in the mass spectrum.

The first two functions are employed in many common analytical separation methods. The third function is associated with the most modem MS-techniques in combination with the newest computer-aided database search programs, which allow for the identification of a protein in dependence on the amino acid sequence of one single peptide or a few peptides (e.g. cysteine-containing peptides).

The advantages of this method are obvious: Each available amount of starting material can be processed. Also proteins only present in minimal amounts are detectable and quantifiable, since they are enriched by means of a cysteine-specific selection. By means of other amino acid specific or substrate specific functional groups in the MeCAT reagent, further proteins can be reliably determined in the analysis. The complexity of the peptide mixture is reduced this way, thus allowing for a largely reduced expenditure of work and a more rapid and successful protein identification via data base search programs.

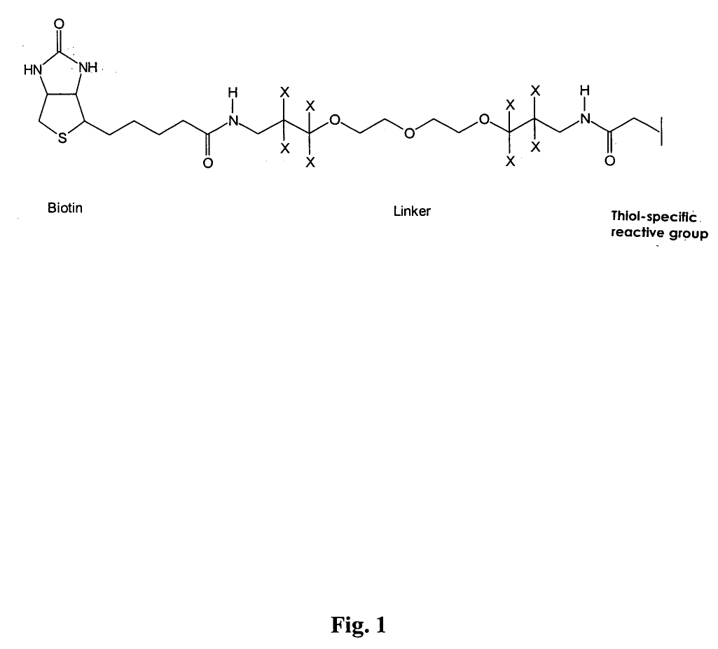

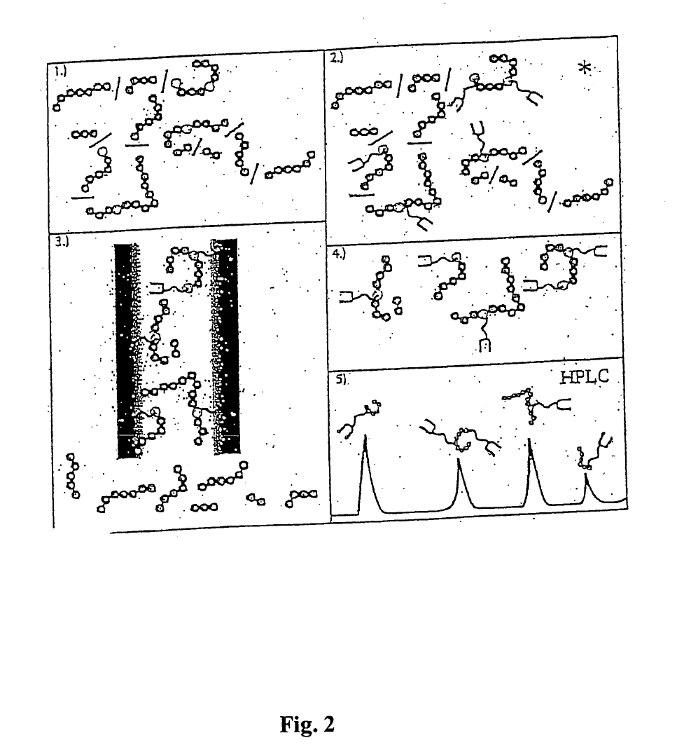

Instead of an isotope label, the present invention provides the integration of a metal chelate complex into the reagent as an alternative. A concept, how these reagents may be designed, is illustrated in the FIGS. 1 and 2.

The synthesis of an isotope-labeled linker is very expensive and not always possible, since, as it is generally known, there only is a very limited number of stable isotope reagents such as ²H, ¹³C or ¹⁵N. This e.g. means that samples derived from a very limited number of cell cultures, which have been exposed to different conditions, can be investigated and compared in respect to the quantitative and qualitative detection of dynamic changes in protein production. Literature describes examples for the comparison between two samples with ¹H- and ²H-labelled ICATs.

Metal ions are available in a much greater variety and at lower price. It just depends on finding suitable ones and packing them appropriately into the amino acid specific reagent thereby preventing them from getting lost by dissociation or exchange reactions.

The candidate chelate ligand must stabilize the metal ion well enough that the complex remains completely intact during the entire process, that its stability is ensured also in case of larger pH changes, and that no exchange processes with the peptides as potential ligands can happen. The solubility characteristics of the complex are not allowed to largely differ from those of the other components of the reagent, i.e. the protein-reactive functional group and the molecule portion for chromatographic separation purposes. The entire molecule preferably has to be soluble in the sample solution in order to ensure an efficient interaction of the tag with the specific protein binding sites.

For quick and unambiguous protein identification, one can integrate into the protein-reactive reagent a metal ion, which normally is not found in proteins and which has a very characteristic isotope pattern. Such a metal ion will be very easily detected in the mass spectrum of the labeled peptide. Computer algorithms can automatically compare the experimentally observed isotope pattern of the mass fragment, with or without the metal ion or mass specific labeling. This causes no greater demand on the sensitivity of the employed mass spectrometer. In contrast, the complexing agents bound to the peptides will positively affect the sensitivity of detection, since complex forming agents are known as strongly contaminating substances in the mass spectrometry of peptides, thus normally requiring to avoid them even at the lowest concentrations. Via the automatic screening of the mass spectra of all peptides separated by 2DLC or another suitable method, it should be possible to very rapidly and unambiguously select the cysteine-containing peptides for reason of their isotope pattern from a peptide mixture mainly containing peptides without cytosine residues. Only the exactly determined mass of these selected peptides is used for protein identification by correlating the experimental data with the data from genome and protein databases. The sequencing of peptides by CID-MS allows for the identification.

For the relative protein quantification and characterization in several protein samples, several metal chelate complexes with identical ligand portion, but with different metal ions come into question, wherein these complexes have to be such resemblant in respect to their thermodynamic stability and their kinetic behavior that metal exchange processes between them can be ruled out. The relative atomic masses of the metal ions should not differ by more than 10 Daltons in order to detect matching peptide pairs easily in the mass spectrum. The metal ions moreover should be low in isotopes in order to avoid unnecessary complication of the assignment. Besides the protein-reactive functional group, the metal ion specific reagent may comprise a molecular component for separating the labeled peptides after protein hydrolysis by means of column chromatography. FIG. 1 schematically illustrates the preferred strategy for quantifying the protein expression by means of metal specifically labeled reagents (MeCATs/MeCODs).

For the efficient binding of the metal ion, macrocyclic ligands are particularly suitable as chelates, since they are characterized by a high thermodynamic stability and a kinetically inert behavior in respect to dissociation. For reason of their topological characteristics, the macrocycles provide a multiplicity of strategically distributed donor atoms, which, in case of a suitable conformation and dimension of the ligand can interact in an effective manner with the metal ion. A “statistical stabilization” follows from the very low probability of a simultaneous break up of all metal-ligand-donor-bonds. Similar to the receptor binding sites of enzymes, a large number of coordinate interactions, which are only weak as single interactions, lead to a binding of the metal ion, which, in case of a suitable molecular architecture, is not just stable, but also selective. Thereby, in contrast to ligands with open ligand chains, the exchange with biologically relevant metal ions is effectively prevented (see table 1).

The present invention relates to a method and a reagent suitable for performing said method, which method includes a reproducible, systematic, qualitative and quantitative proteome characterization by means of non-isotope metal coded markers and—among other items—the most modem tandem methods of mass spectrometry.

The metal code is a macrocyclic lanthanoid chelate complex on the basis of DOTA (1,4,7,10-tetraazacyclododecane-1,4,7,10-tetraacetic acid) or a transition metal complex on the basis of NOTA (1,4,7-triazacyclononane-1,4,7-triacetic acid), which has to be equipped with an amino acid specific functional group and a further molecular component for the chromatographic separation of the labeled peptides. The marker to be synthesized must display a good solubility in water and a high kinetic stability. Compounds with different lanthanoid ions must not significantly differ in respect to their chemical reactivity and physical properties. The metal coded markers are characterized in respect to their structure, their thermodynamic properties in aqueous solution and their reactive behavior towards peptides. Their reproducible application in proteome analysis has to be tested in model experiments and real samples in combination with multidimensional chromatography, MS/MS and database analysis.

The metal coded markers are covalently bound to the proteins of cell lysates in a “site-specific” manner. After the proteolysis of the proteins, the metal labeled peptides are isolated chromatographically and are further fractionated in order to be then quantified by mass spectrometry and, in the second step, to be sequenced. By means of a direct quantitative comparison of well determined states, one features differences in the protein composition, which then have to be correlated with biological effects.

In combination with a data base search, it is possible to identify the basal proteins of interest via one single or just a few peptides.

In this field of coordination chemistry, there exist lots of works from the last 15-20 years, the disclosure of which can be readily resorted to in the context of the present invention (see table 1).

The cyclic ligand, which preferably may be a functionalized tetraaza-macrocycle, i.e. a derivative of DOTA (1,4,7,10-tetraazacyclododecane-1,4,7,10-tetraacetic acid) or a triaza-macrocycle, a derivative of NOTA (1,4,7-triazacyclononane-1,4,7-triacetic acid), is either constructed of amino acids, or synthesized according to a very efficient, template-assisted cyclization reactions, which has been recently developed in the applicants' work group. The protein-reactive group and the function for peptide isolation (e.g. a specific amino acid for the covalent binding to a column containing isothiocyanate groups or biotin for affinity chromatography) can be integrated into the carbon scaffold of the macrocyclic ligand, or the metal chelate complex is suitably connected with the protein-reactive group and the function for peptide isolation via a linker.

Suitable as metal ions for the NOTA-ligand are transition metal ions like copper(II), nickel(II) and zinc(II).

Suitable metal ions for the DOTA-like ligands are the lanthanoid ions, which will form very stable complexes with comparably high complex stability constants and very similar molecular weights with this class of ligands (see table 2). They are very similar in respect to their chemical properties, and the contraction of the ion radius in consequence of the mass increase in case of the very well studied lanthanoid-DOTA-complexes (DOTA=1,4,7,10-tetraazacyclododecane-1,4,7,10-tetraacetic acid) only has a minute influence on the kinetic stability of the lanthanoid chelate complexes. The high in vivo stability of these compounds led to the successful employment of the DOTA-gadolinium(III)-complex as a contrasting agent in magnetic resonance tomography. For in vivo applications, the kinetic stability of the complex is much more important than their thermodynamic stability. An inert complex will not show any ligand or metal exchange reactions, even when the thermodynamic stability constant is not very high. One reason for the DOTA-lanthanoid-complexes being both very stable and inert, is the optimal size relation between the metal ion and the cavity provided by the ligand. Metal ion and ligand constitute a fixed, well locked structure, which under physiological conditions shows an extremely slow dissociation and can only be attacked by protons when being in an acidic medium. [Gd(DOTA)(H₂O)]⁻ shows a half-life of 200 days in an aqueous solution at pH 5 and a half-life of 85 days at pH 2. The well investigated metal exchange reaction between [Gd(DOTA)]⁻ and [Eu(DOTA)]⁻ in the pH-range 3.2-5.0 shows, that the velocity determining step of this exchange reaction is the proton-assisted dissociation of [Gd(DOTA)]⁻. Even when a protonation takes place at the acetate groups, these mono- and di-protonated complexes are not reactive, since the metal ion is located within the coordination cage. To achieve destruction of this cage, the protons have to be transferred to the N-atoms. This process only takes place extremely slowly via a rearrangement of the entire complex. On the basis of this investigation, metal exchange reactions between DOTA-lanthanoid complexes in the relevant time interval and pH range can be excluded with a very high probability.

TABLE 1
Stability constants, LD₅₀-rate and selectivity factor (Kₛₑₗ)
for selected ligands.

| | | Log | Log | Log | Log | Log |
|---|---|---|---|---|---|---|
| Ligand | LD₅₀^{a} | Kₛₑₗ | K_{GdL} | K_{Cal} | K_{CuL} | K_{ZnL} |
| EDTA | 0.3 | 4.23 | 17.7 | 10.61 | 18.78 | 16.5 |
| DTPA-BP | 2.8 | 5.32 | 16.83 | | | |
| DTPA | 5.6 | 7.04 | 22.46 | 10.75 | 21.38 | 18.29 |
| DOTA | 11 | 8.3 | 24.6 | 17.23 | 22.63 | 21.05 |
| DO3A | 7-9 | 4.13 | 21.0 | 11.74 | 22.87 | 19.26 |
| HP-DO3A | 12 | 6.95 | 23.8 | 14.83 | 22.84 | 19.37 |
| DTPA- | 17.8 | 9.04 | 16.85 | 7.17 | 13.03 | 12.04 |
| BMA | | | | | | |

^{a}Intravenous LD₅₀-rate in mice, mmole/kg

TABLE 2
Stability constants (logK) of LnDOTA-complexes

| | Relative | | | |
|---|---|---|---|---|
| | atomic | | | |
| | Log mass | K_{LnDOTA} Log | K_{LnDOTA} Log | K_{LnDOTA,} |
| | [g/mole] | 1 M NaCl, 37° | 0.1M KCl, 25° | other works |
| La | 138.91 | 20.7 | 22.9 | 21.7 (0.1 M KCl, |
| | | | | 25°) |
| Ce | 140.12 | 21.6 | 23.4 | |
| Pr | 140.91 | 22.4 | 23.0 | |
| Nd | 144.24 | 22.5 | 23.0 | |
| Sm | 150.36 | 23.3 | 23.0 | |
| Eu | 151.97 | 23.7 | 23.5 | 28.2 (1M NaCl, |
| | | | | 20° C.) |
| Gd | 157.25 | 23.6 | 24.7 | 22.1 (1M NaCl, |
| | | | | 25° C.); 24.0 (0.1 M |
| | | | | KCl, 25° C.) |
| Tb | 158.93 | 23.6 | 24.7 | 28.6 (1M NaCl, |
| | | | | 20° C.) |
| Dy | 162.50 | 23.5 | 24.8 | |
| Ho | 164.93 | 23.5 | 24.5 | |
| Er | 167.26 | 23.5 | 24.4 | |
| Tm | 168.93 | 23.7 | 24.4 | |
| Yb | 173.04 | 24.0 | 25.0 | |
| Lu | 174.97 | 23.5 | 25.4 | 29.2 (1M NaCl, |
| | | | | 25° C.) |

The development of macrocyclic, lanthanoid-specific ligands experienced a remarkable advance since the beginning of the 80ies for reason of their successful medical employment both in therapy and diagnostics. A review of Lauffer et al. being published in 1999 deals with Gd(III)-chelates as contrasting agents for magnetic resonance tomography (MRT) and impressively summarizes the most important research results of the last decade.

An important aspect of the present invention is the synthesis, characterization and use-directed investigation of binuclear macrocyclic lanthanoid chelate complexes, which have been conceived as potential MRI contrasting agents for medical diagnostics. In contrast to the very well investigated lanthanoid complexes with the ligand DOTA (1,4,7,10-tetraazacyclododecane-1,4,7,10-tetraacetate) and compounds deduced from DOTA, there at present exits only a very limited number of known multinuclear macrocyclic lanthanoid chelate complexes with a very good water-solubility and water-stability.

Having this aim, we succeeded to synthesize two ligands, 1,4,7,10,13,16,19,22-octaazacyclotetracosane-1,4,7,10,13,16,19,22-octaacetic acid (OTEC), and 1,4,7,10,14-17,20,23 -octaazacyclohexacosane-1,4,7,10,14,17,20,23-octaacetic acid (OHEC), which are able to form mono- and binuclear lanthanoid complexes. There existence was successfully detected by means of FAB-mass spectrometry. As a highlight in coordinative chemistry, the determination of the solid-state structures of the binuclear chelate complexes of the OHEC-ligand (Ln=Y, La, Eu, Gd, Th, Yb, Lu), was achieved by means of X-ray structural analysis. Besides the structural information, the X-ray analyses provides hints about the number of water molecules coordinated within the first coordination sphere, which essentially contribute to the efficiency as a contrasting agent. We have discovered, that the dimension of the ion radius of the metals crucially co-affects the conformation of the ligand in the complex and thus also the properties as a MRI-contrasting agent.

By means of dynamic NMR-measurements, we have investigated the complexes' conformation equilibriums in solution. For the yttrium and europium complexes of OHEC, the determination of successful synthesis was additionally accomplished by means of one- and two-dimensional NMR-methods. The mono- and binuclear europium complexes with OHEC allowed to be successfully further characterized by polarography. The determination of the relaxivity of the Gd-complexes was accomplished by NMRD-measurements (nuclear magnetic relaxation dispersion). We have determined relaxivities, which are significantly higher than those of the contrasting agents used in the clinical field. Therefore, we have a reasonable hope to have found a new class of potential contrasting agents with improved characteristics for medical diagnostics.

The invention will now be further illustrated by means of examples and with reference to the accompanying figures, without being limited to them. What is shown is in:

FIG. 1: the structure of an exemplary reagent used in the MeCAT. X is either a H or a chelate group.

FIG. 2: the schematic depiction of a MeCAT-method with 1) enzymatic cleavage; 2) coupling to the MeCAT-reagent; 3) selection of the labeled peptides; 4) elution of the selected peptides; 5) separation of the labeled peptides; subsequently mass spectrometric analysis. * Here, it is possible to pool the samples A and B.

### EXAMPLES

Synthesis Planning—Preparatory Works

Aim of this synthesis is the preparation of a double C-substituted tetraaza-macrocycle. Starting from an amino-protected lysine-hydroxysuccinimide ester (2), the di-lysine-derivative (3) being internally connected by a peptide bond is obtained by reacting two equivalents of said ester (2) with one equivalent of ethylene diamine. Removing the protection from two amino functions provides the one component for the cyclization reaction, the mesylation of ethylene glycol provides the other component. In a subsequent [1+1]-cyclocondensation, one obtains the double C-substituted tetraazadicarbonyl-cycle (4). By reducing the two carbonyl-functions, one finally obtains the tetraaza-cycle (1), which can be provided with further functions at both side chains. 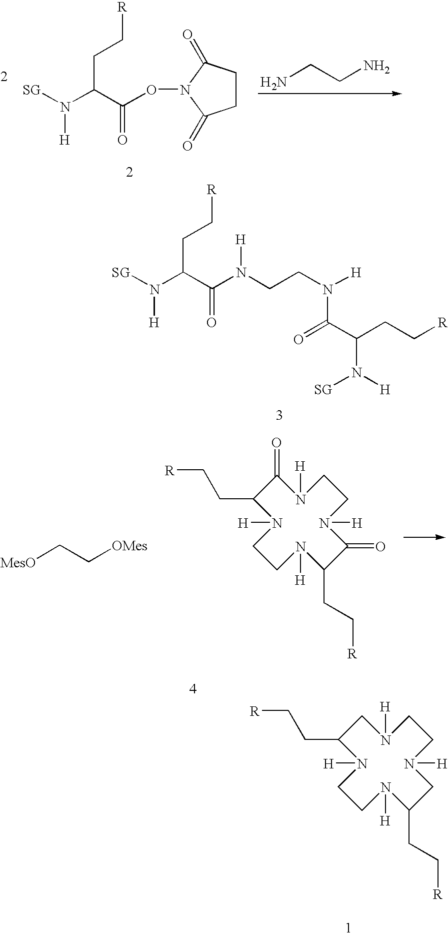
Concept of the Experiment:

Aim of these experiments was the synthesis and application of new functional markers for the identification and quantification of cell proteins. These markers should 1. allow to be bound to specific amino acid groups of denatured proteins, 2. allow to be isolated from a large peptide pool by means of affinity chromatography and other separation methods, and 3. allow for an identification and quantification of the basal proteins on the basis of the labeled peptide fragments by using mass spectrometry and data base analysis.

For this aim, the following elements had to be combined in one molecule:

1. An amino acid specific or sulfhydryl specific group, like e.g. an amine-reactive pentafluorophenyl ester group, an amine-reactive N-hydroxysuccinimide ester group, sulfonylhalide, isocyanate, isothiocyanate, active ester, tetrafluorophenyl ester, an acid halide and an acid anhydride, a homoserine lactone-reactive primary amine group and a carboxylic acid-reactive amine, alcohol or 2,3,5,6-tetrafluorophenyltrifluoro-acetate, a iodine acetylamide group , an epoxide, an α-haloacyl group , a nitrile, a sulfonated alkyl , an arylthiol or a maleimide, which selectively reacts with a functional group in the protein, in this example with SH-groups in the cysteine, or a functional group interacting with a protein binding site (ligand-receptor interaction).

2. Reactive groups for binding to a support material (e.g. for binding the complex to a column material with subsequent binding to peptides) or to biotin or other molecules known from affinity chromatography, which were coupled to a corresponding counter-reagent in order to allow for the separation of the labeled peptides, wherein these groups/molecules allowed to be readily cleaved off again from the remaining molecule after the separation step, wherein the reactive groups can be selected from the acid halides, aldehydes, isocyanate derivatives, succinimide derivatives, imidazolyl carbamate derivatives, Traut's reagent-derivatives, sulfonic acid chloride derivatives, oxirane derivatives, imidates, hydrazines, sulfosuccinimidyl derivatives, diimide derivatives, maleimide derivatives and 7-sulfobenzofurazane derivatives.

3. The essential part of the novel markers, a macrocycle forming metal complexes of high kinetic and thermodynamic stability.

These markers were tested for their applicability in the proteome analytics making use of the performance of modern mass spectrometry. For this aim, we used a test mixture of 5-10 proteins and a number of “real life samples”.

General Approach

The essential part of the proposed markers are macrocycles on the basis of polyaza-poly-acetic acids (DOTA/NOTA), the metal complexes of which have the required stability. These macrocycles had to be synthesized in sufficient amounts, thereby providing them with one or two coupling sites for further marker elements or coupling the macrocycles via a suitable linker with the remaining MeCAT-components. The preferred method starts from amino acids, wherein the introduction of the MeCAT-components is accomplished by coupling them to C-atoms of the macrocycle. In an alternative method, the macrocycle is connected with the remaining MeCAT-components by means of a suitable linker. In a third method, the synthesis is accomplished according to solid phase peptide synthesis in a peptide synthesis device with subsequent intramolecular closure of the tosylamide ring. The MeCAT reagents were again coupled to C-atoms of the aza-macrocycle. The purification of the peptides was accomplished by means of preparative HPLC.

The ligands were complexed with trivalent lanthanoid ions, extensively characterized and tested for their applicability as a MeCAT-reagent with the desired properties. The following demands were made for the reagent:
The complexes must have sufficient kinetic stability, i.e. metal exchange reactions should be negligibly small.
This coding technique with different metal ions served as an internal standard method in order to determine the relative concentration of the differently labeled components from different samples. Therefore, the chemical and physical properties of the MeCAT-reagents with different metal ions had to be identical the most possible—among other things—in respect to the reaction with the proteins and their chromatographic separation behavior.
The molar mass should not largely exceed that of the ICAT-reagent.

The following investigations were then performed:

a) Characterization of the MeCAT-reagents by means of MS and NMR;

b) Test of the amino acid specific or substrate specific binding properties of the MeCAT-reagents and behavior of the labeled peptides in the mass spectrometer by using a small substance pool comprising about 10 peptides;

c) Systematic investigation and optimization of the behavior of the labeled and unlabelled peptides in affinity chromatography and other chromatographic separation methods (ion exchange chromatography, RP-chromatography), in particular investigation of the reproducibility and the recovering rate of the labeled peptides;

d) Verification of the reproducibility of the relative concentration conditions (determined by suitable methods of mass spectrometry) of the peptides being labeled with different metal ions but being chemically identical for the rest by relying on the relative signal/intensity-ratio of the respective matching peptide peaks in the mass spectrum;

e) Investigation of the properties of the MeCAT-reagents in real samples.

Preparation of Suitable Linkers and Their Coupling to the MeCAT-Components

a) The linker was coupled to a biotin unit for its binding to avidin.

b) The linker was coupled to glycine for its covalent binding to a chromatographic column via isothiocyanate groups. In order to allow the unlabelled peptides to leave the column in an unrestricted manner, it was necessary in this case to previously derivatize all amino groups with phenylisothiocyanate.

c) The linker was coupled to a iodine acetic acid unit for the selective labeling of cysteine-containing peptides.

d) The linker was coupled to a succinic acid anhydride unit for the labeling of amine-containing peptides.

### REFERENCES

F. Sanger, Nature 1977, 265, 687.
V. C. Wasinger, S, J. Cordwell, A. Cerpa-Poljak, J. X. Yan, A. A. Gooley, M. R. Wilkins, M. W. Duncan, R. Harris, K. L. Williams, I. Humphery-Smith, Elektrophoresis 1995,16, 1090.
J. E. Celis, Electrophoresis 1990, 11, 989.
- K. B. Mullis, F. A. Faloona, Methods Enzymol. 1987, 155, 335.
A. J. Link, Electrophoresis 1997,18, 1314.
A. e. a. Shevchenko, Proc. Natl. Acad. Sci. USA 1996, 93, 1440,
D. Figeys, Electrophoresis 1998, 19, 1811.
D. R. e. a. Goodlett, Anal. Chem. 2000, 72, 1112.
K. L. Williams, Electrophoresis 1999, 20, 678.
M. Quadroni, P. James, Electrophoresis 1999, 20, 664.
I. Humphery-Smith, S. J. Cordwell, W. P. Blackstock, Electrophoresis 1997, J8, 1217.
R. H. Aebersold., M. H. Gelb, S. P. Gygi, C, R. Scott, F. Turecek, S. Gerber, B. Rist, in PCT/US99/19415.
P. S. Gygi, B. Rist, S. A. Gerber, F, Turecek, M. H. Gelb, R. Aebersold, Nature Biotechnology 1999, 77, 994.
D. F. Hunt, J. R. Yates, J. Shabanowilz, S. Winston, C. R. Hauer, Proc. Natl. Acad. Sci. USA 1986, 83, 6233.
C. S. Spahr, S. A. Susin, E. J. Bures, J. H. Robinson, M- T. Davis, M. D. McGinley, G. Kroemer, S. D. Patterson, Electrophoresis 2000, 21, 1635.
S. P. Gygi, B. Rist, T. J. Griffin, J. Eng, R. Aebersold, Journal of Proteome Research 2002.
T. J. Griffin, D. K. M. Han, S. P. Gygi, B. Rist, H. Lee, R. Aebersold, K. C. Parker, Journal of the American Society for Mass Spectrometry 2001, 12, 1238.
B. J M. B. Smolka, H. Zhou, S. Purkayastha, R. Aebersold, Anal. Biochem. 2001, 297, 25
D. K. Han, J. Eng, H. Zhou, R. Aebersold, Nature Biotechnology 2001, 19, 946.
T. J. Griffin, S. P. Gygi, B. Rist, R. Aebersold, A. Loboda, A. Jilkine, W. Ens, K. G Standing, Anal. Chem. 2001, 73, 978.
R. Zhang, F. E. Regnier, Journal of Proteome Research 2002.
X. Wang, T. Jin, V. Comblin, A. Lopez-Mut, M- E., J. F. Desreux, Inorg. Chem. 1992, 31, 1095.
G. R. Choppin, K. M. Schaab, Morg. Chim Acia 1996, 252, 299.
A. E. Martell, R. J. Motekaitis, E. T. Clarke, R. Delgado, Y. Sun, R. Ma, Supranolecular Chemistry 1996, 6, 353.
R. Delgado, J. J. R. Frausto Da Silva, Talanta 1982, 29, 815.
E, Toth, E. Briicher, I. Lazar, I. Toth, Inorg. Chem. 1994, 33, 4070.
P. Caravan, J. J. Ellison, T. J. McMuny, R. B. Lauffer, Chem. Rev. 1999, 99, 2293.
W. P. Cacheris. S. C. Ouay, S. M. Rocklage, Magnetic Resonance Imaging 1990, 8, 467.

1. A method for the identification and quantification of one or more proteins in a sample containing a mixture of proteins, wherein said method comprises the steps of:
a) providing a sample which contains a mixture of proteins;
b) providing a reagent for the analysis of peptides wherein the reagent has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding to peptides or other biomolecules to be analyzed;
c) cleaving the proteins in the sample in order to produce peptides;
d) coupling the peptides to the reagent of step b);
e) selecting the peptides labeled in step d) under the employment of a functional group for the reversible, covalent or non-covalent binding to a support material and removal of the unbound peptides;
f) releasing the bound peptides from the support material and elution from the matrix;
and
g) detecting and identifying the labeled peptides by means of mass spectrometry.

2. The method, according to claim 1, wherein the cleavage of the peptides is performed enzymatically or chemically.

3. The method, according to claim 1, wherein the labeled peptides, after their release from the support material and before their analysis by mass spectrometry, are separated from each other by means of HPLC.

4. The method, according to claim 1, characterized in that several protein- and/or peptide-containing samples are analyzed together.

5. The method, according to claim 1, further comprising the sequencing of the labeled peptides.

6. A method for the detection of the relative expression of proteins in a protein-containing sample, wherein said method comprises the steps of:
a) providing a biological sample which contains proteins;
b) providing a reagent for the analysis of peptides wherein the reagent has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding to peptides or other biomolecules to be analyzed;
c) cleaving the proteins in the sample in order to produce peptides;
d) coupling the peptides to the reagent of step b);
e) selecting the peptides labeled in step d) under the employment of a functional group for the reversible, covalent or non-covalent binding to a support material and removal of the unbound peptides;
f) releasing the bound peptides from the support material and elution from the matrix;
g) detecting and identifying the labeled peptides by means of mass spectrometry; and
h) measuring the relative occurrence of the differently labeled peptides as distinct peaks of ions in order to determine the relative expression of the protein, from which the affinity-labeled peptide is derived.

7. The method, according to claim 6, characterized in that the arrangement of the groups A, Y and PRG is interchanged.

8. The method, according to claim 6, characterized in that the labeled peptides are detected by means of a tandem technique selected from the group consisting of matrix-assisted laser desorption/ionization (MALDI), time-of-flight (TOF)-TOF-MS and electrospray ionization (ESI)-MS.

9. A reagent for the mass spectroscopic analysis of peptides which has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding of peptides or other biomolecules to be analyzed.

10. The reagent, according to claim 9, wherein the arrangement of the groups A, Y and PRG is interchanged.

11. The reagent, according to claim 9, wherein the PRG is selected from the group consisting of sulfhydryl-reactive groups, amine-reactive groups and enzyme substrates.

12. The reagent, according to claim 11, wherein the PRG is selected from the group consisting of amine-reactive pentafluorophenyl ester groups, amine-reactive N-hydroxysuccinimide ester groups, sulfonylhalides, isocyanates, isothiocyanates, active esters, tetrafluorophenyl esters, acid halides and acid anhydrides, homoserine lactone-reactive primary amine groups and carboxylic acid-reactive amines, alcohols or 2,3,5,6-tetrafluorophenyltrifluoro-acetates, iodine acetylamide groups, epoxides, α-haloacyl groups, nitriles, sulfonateds alkyls, arylthiols and maleimides.

13. The reagent, according to claim 9, wherein A is selected from the group consisting of biotin or modified biotin, 1,2-diols, glutathiones, maltoses, nitrilotriacetic acid groups, oligohistidines and haptens or other reactive reagents allowing for a reversible binding to a support material.

14. The reagent, according to claim 9, further comprising a linker between the groups A, Y and/or PRG, which is cleavable in a chemical and/or enzymatic way and/or by exposure to radiation or light.

15. The reagent, according to claim 14, wherein the linker contains a disulfide group.

16. The reagent, according to claim 9, wherein Y is selected from the group consisting of macrocyclic lanthanoid chelate complexes, functionalized tetraaza-macrocycles, polyaza-polyacetic acids, DOTA, DOTA-derivatives, NOTA, NOTA-derivatives, 1,4,7,10,13,16,1 9,22-octaazacyclotetracosane-1,4,7,10,13,16,1 9,22-octaacetic acid (OTEC), 1,4,7,10,14-17,20,23-octaazacyclohexacosane-1,4,7,10,14,17,20,23-octaacetic acid (OHEC), EDTA, DTPA-BP, DTPA, DO3A, HP-DO3A and DTPA-BMA.

17. The reagent, according to claim 9, wherein the metal bound by the chelate complex is selected from the group consisting of Ag, Al, As, Au, Be, Cd, Ce, Co, Cr, Cu, Dy, Er, Eu, Fe, Gd, Hg, Ho, In, La, Li, Lu, Mn, Na, Nd, Ni, Pb, Pr, Rb, Rd, Sb, Sm, Sn, Th, Tl, Tm, V, W, Y, Yb and Zn.

18. The reagent, according to claim 9, wherein the chelate forming group is labeled with several different metals.

19. A method for detecting peptides in a biological sample and/or for determining the relative expression of proteins in a protein-containing sample wherein said method comprises the use of a reagent for the mass spectroscopic analysis of peptides which has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding of peptides or other biomolecules to be analyzed.

20. A method for the diagnosis of diseases of an animal by detecting the relative expression of proteins in a protein-containing sample taken from the animal wherein said method comprises the use of a reagent for the mass spectroscopic analysis of peptides which has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding of peptides or other biomolecules to be analyzed.

21. A diagnostic kit, containing a reagent for the mass spectroscopic analysis of peptides which has the general formula
A-Y—PRG
in which
A constitutes at least one functional group for the reversible, covalent or non-covalent binding to a support material,
Y is a group comprising at least one chelate function for metals being low in isotopes, and
PRG is a reactive group for the selective binding of peptides or other biomolecules to be analyzed;
wherein said kit further comprises additional substances and/or enzymes suitable for the detection of peptides in a biological sample and/or the determination of the relative expression of proteins in a protein-containing sample, in particular containing an internal standard.
